(19) United States
(12) Patent Application Publication
(10) Pub. No.: US 2007/0055133 A1
(43) Pub. Date: Mar. 8, 2007

(54) Input device of wireless communication terminal using biomagnetism measurement

(75) Inventor: Han-Sang Lim, Seoul (KR)

Correspondence Address: DILWORTH & BARRESE, LLP, 333 EARLE OVINGTON BLVD., UNIONDALE, NY, 11553, US

(73) Assignee: Samsung Electronics Co., Ltd., Suwon-si (KR)

(21) Appl. No.: 11/511,992

(22) Filed: Aug. 29, 2006

(30) Foreign Application Priority Data
Aug. 29, 2005 (KR) 2005-79686

(51) Int. Cl. A61B 5/05

(52) U.S. Cl. 600/409

(57) ABSTRACT

Provided is an input device of a wearable wireless communication terminal. The device includes a biomagnetic sensing unit having a plurality of sensors for sensing biomagnetism caused by human muscular motion, a distinguishing unit for generating a signal(s) for distinguishing whether one or more sensing signals are inputted from any one(s) of the plurality of sensors of the biomagnetic sensing unit, a memory for storing a key mapping algorithm, and a controller for combining one signal or at least two signals outputted from the distinguishing unit, depending on the key mapping algorithm, and recognizing the combined signal as key input.

### PRIORITY

This application claims priority under 35 U.S.C. § 119 to an application entitled “Input Device of Wireless Communication Terminal using Biomagnetism Measurement” filed in the Korean Intellectual Property Office on Aug. 29, 2005 and assigned Serial No. 2005-79686, the contents of which are incorporated herein by reference.

### BACKGROUND OF THE INVENTION

1. Field of the Invention

The present invention relates generally to a wearable wireless communication terminal, and in particular, to an input device for enabling key recognition through biomagnetism measurement.

2. Description of the Related Art

Human muscle or nervous tissue generates an electric current by its cell activity. The electric current generates a magnetic field that is called biomagnetism. Biomagnetic fields refer to magnetic signals generated from the human heart, brain, spinal cord and stomach. The magnetic signals can be measured using a Superconducting Quantum Interference Device (SQUID).

Biomagnetism measurement technology is a technology for measuring spatial distribution of the magnetic field outside a human body using a multi-channel SQUID, and dynamically indicating information (position, direction, and intensity variation) on active current depending on time. By electrical activity within the human body, the magnetic field is generated. Since the human body is transparent for the magnetic field, the magnetic field can be measured at a position spatially remote from a magnetic field generating source.

Diagnosis using the biomagnetism measurement is neither tactile nor destructive, and makes possible accurate measurement of minute variations of the active current generated within the brain or the heart, due to excellent temporal and spatial resolution. Therefore, the diagnosis is a next generation medical diagnosis technology of importance for use in researching a brain (heart) function and diagnosing a functional disease.

The SQUID, a high sensitivity magnetic field sensing unit using Josephson effect, has a sensitivity of several fT. Therefore, the SQUID can be used to measure signal transmission in the muscle or nervous tissue.

A portable telephone is a primary example of a wireless communication terminal. It generally includes an input device with a keypad constituted of button keys or a touch screen pad provided on a liquid crystal display. A user presses keys or touches a screen through the input device to input a desired numeral or character and request execution of a specific function.

In the keypad input device, the key should occupy a sufficient area such that it is not difficult for the user to press the key. This limits the construction of the telephone, and makes it more difficult for a user to wear the telephone. In other words, since the user always needs to see the keypad in order to press the key to input the desired numeral or character, the user's vision of the keypad is limited during key press by his/her hands as the key area is pressed. As the telephone has become increasingly diverse in function, more keys are being required. However, due to the limitations of the keypad, individual keys (or key combinations) are being made multi-functional. As a result, menus are becoming more and more complex.

### SUMMARY OF THE INVENTION

It is, therefore, an object of the present invention to provide an input device of a wearable wireless communication terminal, for enabling key recognition through measurement of biomagnetism caused by human muscular motion.

To achieve the above and other objects, there is provided an input device of a wearable wireless communication terminal, including a biomagnetic sensing unit having a plurality of sensors for sensing biomagnetism caused by human muscular motion, a distinguishing unit for generating at least two signals for distinguishing whether one or more sensing signals are inputted from at least one of the plurality of sensors of the biomagnetic sensing unit, a memory for storing key mapping algorithm, and a controller for combining the at least two signals outputted from the distinguishing unit, depending on the key mapping algorithm, and recognizing the combined signal as key input.

### BRIEF DESCRIPTION OF THE DRAWINGS

The above and other objects, features and advantages of the present invention will become more apparent from the following detailed description when taken in conjunction with the accompanying drawings in which:

FIG. 1 illustrates a construction of an input device of a wireless communication terminal using biomagnetism measurement according to of the present invention.

### DETAILED DESCRIPTION OF THE PREFERRED EMBODIMENT

A preferred embodiment of the present invention will now be described in detail with reference to the annexed drawings. In the drawings, the same or similar elements are denoted by the same reference numerals even though they are depicted in different drawings. In the following description, a detailed description of known functions and configurations incorporated herein has been omitted for the sake of clarity and conciseness.

In the present invention, key input is sensed using the biomagnetism measured by the high sensitivity magnetic field sensing unit.

Signals of the biomagnetic field have very small magnitudes of several fT (10⁻¹⁵ T) to hundreds of fT that are smallest in the brain, tens of fT in a peripheral nerve, and mere ten thousands of fT that are largest even in the heart.

For example, when a wearable telephone is worn on a hand, an input device constituted of a SQUID array is positioned between the telephone and the skin surrounding the hand. If neither a finger nor the hand is in motion, the biomagnetic field generated from the nervous tissue of the hand is varied by about tens of fT, thereby varying a plurality of SQUID output voltages of the SQUID array.

However, such an amount of variation is very little and therefore, cannot be used as it is. So, after the amount of variation is suitably processed, an operation of determining whether the process result is recognized as any key input should be performed.

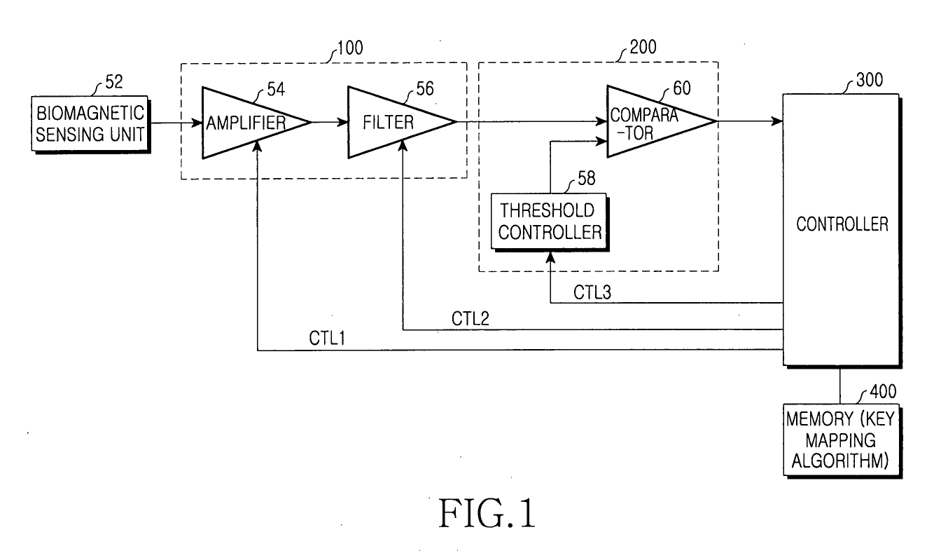

FIG. 1 illustrates a construction of an input device of a wireless communication terminal using the biomagnetism measurement according to the present invention.

A biomagnetic sensing unit 52 senses the biomagnetism caused by human muscular motion. The biomagnetic sensing unit 52 can be embodied using the SQUID array including the plurality of SQUIDs.

A signal processing unit 100 and a determination signal generating unit 200 are distinguishing units. The distinguishing unit is to generate signals for distinguishing whether it receives at least two sensing signals from any one of the plurality of SQUIDs of the biomagnetic sensing unit 52.

The signal processing unit 100 enables the sensing signals outputted from the biomagnetic sensing unit 52 to be suitable to processing in the determination signal generating unit 200. In other words, since the signal outputted from the biomagnetic sensing unit 52 is very weak, the signal processing unit 100 amplifies the very weak signal at a magnitude necessary for the determination signal generating unit 200. The signal processing unit 100 also filters unnecessary components (e.g. noise).

In detail, the signal processing unit 100 includes an amplifier 54 and a filter 56. The amplifier 54 can be a low noise amplifier. The amplifier 54 and the filter 56 are controlled in gain or filter frequency as well as on/off of a corresponding function under predetermined control. Reference symbols CTL1 and CTL2 denote control signal buses applied to the low noise amplifier 54 and the filter 56 from a controller 400, respectively.

For example, assuming that the biomagnetic sensing unit 52 has ten SQUIDs, there can be provided ten of the amplifier 54 and the filter 56, corresponding to the number of SQUIDs. The control signals CTL1 and CTL2 can be determined using suitable bits, depending on on/off bits or control bits for the gain or the filter frequency. In another embodiment, fewer amplifiers and filters can be used, through switching.

The determination signal generating unit 200 receives the sensing signals from the signal processing unit 100, and determines whether it receives the sensing signal of any one of the SQUIDs of the biomagnetic sensing unit 52.

In detail, the determination signal generating unit 200 includes a comparator 60 and a threshold controller 58. The comparator 60 receives the output signal of the signal processing unit 100 at its one input terminal, and receives a signal of the threshold controller 58 at the other input terminal. The comparator 60 determines whether the output signal of the signal processing unit 100 is larger or smaller than a reference threshold, and determines whether it receives the sensing signal of any SQUID. A level of the reference threshold is a value for distinguishing the respective SQUIDs. Since the biomagnetism may be different relative to every person, the level of the reference threshold should be a value based on the biomagnetism. The threshold controller 58 may be comprised of an analog to digital converter (ADC) having an analog output value that varies depending on a digital control signal, and may be constructed such that a pulse width modulation (PWM) signal varies the analog output value through a low pass filter (LPF).

For example, if the biomagnetic sensing unit 52 includes ten SQUIDs, and the signal processing unit 100 also includes ten amplifiers and filters, respectively, the determination signal generating unit 200 will also include ten comparators 60 and threshold controllers 58, respectively. In another embodiment, fewer comparators and threshold controllers can be used.

For example, when a SQUID of a thumb finger contacts a SQUID of an index finger, it is recognized that specific key input is performed, in the determination signal generating unit 200 including one comparator and threshold controller, if ten SQUID outputs and corresponding reference thresholds are sequentially inputted by pair to the two input terminals of the comparator, it can be found that the SQUID of the thumb finger contacts with the SQUID of the index finger.

In another example, the determination signal generating unit 200 can include four comparators and threshold controllers. When the SQUID of each of the thumb fingers of each hand is used as interrupt, default connection to two of the four comparators is performed such that the thresholds corresponding to the respective thumb fingers are provided to the default-connected two comparators. In such a configuration, it is determined whether there is any hand involved, and then, the connection is released. The sensing signals outputted from four SQUIDs of the determined hand are compared with the thresholds of the four comparators, respectively. In this case, the thresholds provided to the respective four comparators correspond to the remaining four fingers of the determined hand.

A control signal CTL3 can be a digital threshold or memory address. The latter is based on the assumption that the threshold controller 58 has the threshold memory (not shown).

The memory 400 stores a key mapping algorithm. The key mapping algorithm is an algorithm for distinguishing the key input based on a biomagnetic signal.

The controller 300 executes the key mapping algorithm, and recognizes the biomagnetism, which is sensed by the biomagnetic sensing unit 52, as the key input. In other words, the controller 300 receives information on whether the determination signal generating unit 200 receives input from any one of sensors of the biomagnetic sensing unit 52, and enables a corresponding function.

For example, when the SQUID array is installed to measure motions of the respective fingers, the controller 300 receives the information on whether the determination signal generating unit 200 receives the input from at least one finger, and performs a function corresponding to an input combination.

In the wireless communication terminal, interrupt pins of devices such as an application processor or a base band supporting a key interface are limited in number. Therefore, for various inputs, specific sensors of the SQUID array can be used as interrupting sensors while the remaining sensors are used as normal. If so, by scanning the remaining sensors input after receiving interrupt, it is possible to identify the combination of interrupting sensors and the remaining sensors, and perform the corresponding functions.

The controller 300 can perform an auto calibration function to adjust the reference threshold level of the threshold controller 58. The controller 300 can perform the auto calibration function of increasing a pulse width of a pulse width modulator or a digital input of the analog to digital converter of the threshold controller 58, in sequence from a small value, measuring a maximal level of each sensor array in absence of finger motion, and adding and storing a margin value.

When the biomagnetic sensing unit 52 is the SQUID array, where the SQUID is positioned at each finger, examples of various inputs will be described as follows.

Bending of any one finger can replace input of a number key on a conventional keypad. Combination of biomagnetic signals measured at the time of bending several fingers can also replace input of a function key on the conventional keypad.

When the thumb finger is bent, it is recognized that a numbered key “1” is inputted. When the index finger is bent, it is recognized that a numbered key “2” is inputted, and when the thumb and index fingers are bent, it is recognized that a call key is inputted. In order to sense the bending of the finger, the biomagnetic sensing unit 52 sets a biomagnetic reference value capable of being sensed in the SQUID of the bent finger, and generates a signal indicating input from the SQUID when the biomagnetic reference value varies more than the reference threshold value. The controller receives the generated signal and determines whether any key input is performed, and performs a mapped function (e.g. input “2”, operation corresponding to the call key).

When the thumb and index fingers are in contact with each other, it is recognized that a numbered key “5” is inputted. In order to sense the contact of the two fingers, the biomagnetic sensing unit 52 sets a biomagnetic reference value capable of being sensed in the SQUID of each of the contact fingers and, when the biomagnetic reference value varies to be more than the reference threshold value, generates signals indicating inputs from the SQUIDs, and the controller determines whether any key input is performed from a combination thereof, and performs a mapped function.

In both cases, the reference threshold level can be automatically calibrated for compensation when the reference threshold level varies depending on a user. The input device can further include a database for storing the threshold level measured on a user-by-user basis in order to compensate for the biomagnetism variance depending on the user.

As described above, in the present invention, the keypad occupying a large area is eliminated from the wearable wireless communication terminal, thereby simplifying a construction of the telephone. Due to the construction simplification, freedom of design can be increased, thereby facilitating design of improving wearability. Since desired input can be performed only with simple operation without finding and pressing a small button of the keypad, the user can not only use the telephone more conveniently, but also can free and his/her eyes and hands of the key input.

While the invention has been shown and described with reference to a certain preferred embodiment thereof, it will be understood by those skilled in the art that various changes in form and details may be made therein without departing from the spirit and scope of the invention as defined by the appended claims.

What is claimed is:

1. An input device of a wearable wireless communication terminal, the device comprising:
a biomagnetic sensing unit having a plurality of sensors for sensing biomagnetism caused by human muscular motion;
a distinguishing unit for generating at least one signal for distinguishing a number of sensing signals that are inputted from any one of the plurality of sensors of the biomagnetic sensing unit;
a memory for storing a key mapping algorithm; and
a controller for combining the at least one signal outputted from the distinguishing unit, depending on the key mapping algorithm, and recognizing the combined signal as key input.

2. The device of claim 1, wherein the distinguishing unit comprises:
a determination signal generating unit for generating at least one signal for determining whether the number of sensing signal is inputted from any one of the plurality of sensors of the biomagnetic sensing unit; and
a signal processing unit for executing processing for changing the sensing signal outputted from the biomagnetic sensing unit into a signal form that is distinguishable by the determination signal generating unit.

3. The device of claim 1, wherein the plurality of sensors are comprised of a Superconducting QUantum Interference Device (SQUID) sensor array.

4. The device of claim 2, wherein the signal processing unit comprises an amplifier for amplifying at least one signal outputted from the biomagnetic sensing unit, at a magnitude necessary for distinguishing by the determination signal generating unit.

5. The device of claim 4, wherein the signal processing unit further comprises a filter for filtering, from the at least one amplified signal, a component that is not necessary for the determination signal generating unit.

6. The device of claim 2, wherein the determination signal generating unit comprises:
a comparator for comparing signals outputted from the signal processing unit with a reference threshold, and providing a determination signal to the controller; and
a threshold controller for providing the reference threshold to the comparator under control of the controller.

7. The device of claim 6, further comprising a database for storing a threshold level measured on a user-by-user basis in order to compensate for a difference of biomagnetism depending on a user.

8. The device of claim 6, wherein at least one sensor of a sensor array is set as at least one interrupting sensor, and key input is mapped by combination of at least one input of the at least one interrupting sensor and inputs of remaining sensors.

9. The device of claim 2, wherein the sensor is detachably attached to a digit, and a selected function of the wireless communication terminal is performed by combination of bending of the digit.

10. The device of claim 2, wherein a function key necessary for the wireless communication terminal is mapped to each portion of an appendage, and input is performed using a biomagnetism difference caused by a motion difference of the mapped portion of the appendage.

11. The device of claim 2, wherein the wireless communication terminal is a wireless telephone.
